(19) United States
(12) Patent Application Publication
(10) Pub. No.: US 2012/0283537 A1
(43) Pub. Date: Nov. 8, 2012

(54) ANALYTE SENSOR LAYERS AND METHODS RELATED THERETO

(76) Inventors: James R. Petisce, San Clemente, CA (US); Henry W. Oviatt, Temecula, CA (US)

(21) Appl. No.: 13/503,745

(22) PCT Filed: Oct. 28, 2010
(86) PCT No.: PCT/US10/54529

§ 371 Date: Jul. 30, 2012

Related U.S. Application Data
(60) Provisional application No. 61/256,505, filed on Oct. 30, 2009.

(51) Int. Cl. A61B 5/1473; G01N 27/28

(52) U.S. Cl. 600/345; 204/403.14; 205/777.5

(57) ABSTRACT

Sensors for measuring an analyte in a subject comprising at least one electrode having an electroactive surface, at least one interferent-reducing layer comprising vinyl ester monomeric units disposed distally from the electroactive surface, an enzyme layer disposed distally from the electroactive surface and a flux-limiting membrane disposed over the at least one interferent-reducing layer and the enzyme layer. Methods of measuring an analyte in a subject comprising providing the electrochemical analyte sensor, contacting a sample comprising an analyte and an interferent with the at least one interferent-reducing layer comprising vinyl ester monomeric units, and measuring the analyte, where the amount of interferent reaching the electroactive surface is reduced.

### TECHNICAL FIELD

The present disclosure relates generally to polymers layers capable of blocking transport of species affecting the performance of analyte sensors and devices comprising same. More particularly, the present disclosure relates to at least one interferent-reducing layer comprising vinyl ester monomeric units capable of blocking transport of such species for analyte detecting devices.

### BACKGROUND

A variety of analyte-measuring devices have been developed in the past few decades for measuring a variety of analytes. Some analyte-measuring devices are substantially continuous devices, while others can analyze a plurality of intermittent blood samples. Some analyte-measuring devices are subcutaneous, transdermal, or intravascular devices, which are typically invasive or minimally invasive, while others are non-invasive in nature. The measurement techniques used by these devices include enzymatic, chemical, physical, electrochemical, spectrophotometric, polarimetric, calorimetric, radiometric, and the like, and generally provide an output signal indicative of the concentration of the analyte of interest. The output signal is typically a raw signal that is used to provide a useful value of the analyte of interest to a user, such as a patient or doctor, using the device. Typically, these analyte-measuring devices include a membrane system that functions to control the flux of species such as acetaminophen, hydroxyurea, potassium iodide, isoniazid and other compounds as they may result in or contribute to false sensor readings in the electrochemical analysis of target analytes present in blood. Conventional analyte-measuring devices that use or incorporate a membrane system, however, suffer from a variety of disadvantages.

### SUMMARY

In general, at least one interferent-reducing layer comprising vinyl ester monomeric units positioned distally from the electroactive surface of an electrochemical analyte sensor and sensor assemblies are disclosed and described. Such sensors provide effective blocking of interferent species as well as rapid chemical, electrical and physical equilibrium with their environment, and as a result, provide fast and accurate analyte levels. Such sensors are of particular use in more demanding sensing applications, such as ICU monitoring.

In a first embodiment, an electrochemical analyte sensor is provided. The electrochemical analyte sensor comprises at least one electrode having an electroactive surface. At least one interferent-reducing layer comprising vinyl ester monomeric units is disposed distally from the electroactive surface. An enzyme layer is disposed distally from the electroactive surface. An analyte flux limiting membrane is disposed over the at least one interferent-reducing layer and the enzyme layer.

In a first aspect of the first embodiment, the at least one interferent-reducing layer is more distal from the electroactive surface than the enzyme layer.

In a second aspect of the first embodiment, the enzyme layer is more distal from the electroactive surface than the at least one interferent-reducing layer.

In a third aspect of the first embodiment, the enzyme layer is positioned between at least two interferent-reducing layers, at least one of the interferent-reducing layer comprising vinyl ester monomeric units.

In a forth aspect of the first embodiment, the electrochemical analyte sensor further comprises a hydrophilic polymer layer in direct contact with the electroactive surface.

In a fifth aspect of the first embodiment, the electrochemical analyte sensor further comprises a hydrophilic layer between the electroactive surface and the enzyme layer.

In a sixth aspect of the first embodiment, the electrochemical analyte sensor further comprises a hydrophilic layer between the electroactive surface and the at least one interferent-reducing layer.

In a seventh aspect of the first embodiment, the at least one interferent-reducing layer is a poly (ethylene-co-vinyl acetate).

In an eighth aspect of the first embodiment, the at least one interferent-reducing layer comprise a wt. % vinyl acetate content of about 33 or less.

In a ninth aspect of the first embodiment, the at least one interferent-reducing layer comprise a wt. % vinyl acetate content of about 25 or less.

In a tenth aspect of the first embodiment, the at least one interferent-reducing layer comprise a wt. % vinyl acetate content of about 18 or less.

In an eleventh aspect of the first embodiment, the at least one interferent-reducing layer comprise a wt. % vinyl acetate content of about 12 or less.

In a second embodiment, an electrochemical analyte sensor assembly is provided. The electrochemical analyte sensor comprises at least one electrode having an electroactive surface. A poly (ethylene-co-vinyl acetate) is disposed distally from the electroactive surface, the poly (ethylene-co-vinyl acetate) having a wt. % vinyl acetate content selected from about 12, about 18, about 25, about 33, or mixtures thereof. An enzyme layer is disposed distally from the poly (ethylene-co-vinyl acetate), wherein the enzyme layer comprises a mixture of glucose oxidase and poly-N-vinylpyrrolidone.

In a third embodiment, a method is provided. The method comprises providing the electrochemical analyte sensor with at least one interferent-reducing layer comprising vinyl ester monomeric units, and contacting a sample comprising an analyte and an interferent, whereby the amount of interferent reaching the electroactive surface is reduced.

In a first aspect of the third embodiment, wherein the sample is in vivo intravenous blood of a subject.

In a second aspect of the third embodiment, wherein the electrochemical analyte sensor is disposed in a catheter.

### BRIEF DESCRIPTION OF THE DRAWINGS

FIG. 1 shows an amperometric sensor in the form of a flex circuit having a working electrode coated with a flux limiting membrane according to an embodiment of the invention.

FIG. 2 is a side cross-sectional view of a working electrode portion of a sensor with defined layers as disclosed herein.

FIG. 3 is a side cross-sectional view of a working electrode portion of a sensor with defined layers as disclosed herein.

FIG. 4 is a side cross-sectional view of a working electrode portion of a sensor with defined layers as disclosed herein.

FIG. 5 is a side view of a multilumen catheter with a sensor assembly according to an embodiment of the invention.

FIG. 6 is a detail of the distal end of the multilumen catheter of FIG. 5 according to an embodiment of the invention.

### DETAILED DESCRIPTION

The following description and examples illustrate some exemplary embodiments of the disclosed invention in detail. Those of skill in the art will recognize that there may be numerous variations and modifications of this invention that may be encompassed by its scope. Accordingly, the description of a certain exemplary embodiment is not intended to limit the scope of the present invention.

In general, at least one interferent-reducing layer comprising vinyl ester monomeric units is positioned distally from the electroactive surface of an electrochemical analyte sensor to provide effective blocking/attenuation of interferent species as well as rapid chemical, electrical and physical equilibrium with their environment, and as a result, provide fast and accurate analyte levels. In one aspect, the at least one interferent-reducing layer comprising vinyl ester monomeric units is positioned distally from the electroactive surface but more proximal to the electroactive surface than the enzyme layer and functions to block/attenuate interferents. In this aspect, the layer comprising vinyl ester monomeric units permits passage of hydrogen peroxide from the enzyme layer to the electroactive surface.

In another aspect, the at least one interferent-reducing layer comprising vinyl ester monomeric units is positioned distally from the electroactive surface and more distal from an enzyme layer. The layer comprising vinyl ester monomeric units block/attenuate interferents. The at least one interferent-reducing layer may further comprise interferent scavenging agents (e.g., bioactive agents that can scavenge, bind-up or substantially inactivate interferants), for example, H₂O₂-degrading enzyme, such as but not limited to glutathione peroxidase (GSH peroxidase), heme-containing peroxidases, eosinophil peroxidase, thyroid peroxidase or horseradish peroxidase (HRP). The scavenging agent can act within the interferent-reducing layer comprising vinyl ester monomeric units.

In another aspect, a plurality of layers comprising vinyl ester monomeric units are positioned distally from the electroactive surface with the enzyme layer positioned between the plurality of layers. Either of the plurality of layers effectively block/attenuate interferents. The at least one interferent-reducing layer may further comprise scavenging agents as described above.

### Definitions

In order to facilitate an understanding of the various aspects disclosed and described herein, the following are defined below.

The term “analyte” as used herein refers without limitation to a substance or chemical constituent of interest in a biological fluid (for example, blood) that may be analyzed. The analyte may be naturally present in the biological fluid, the analyte may be introduced into the body, or the analyte may be a metabolic product of a substance of interest or an enzymatically produced chemical reactant or chemical product of a substance of interest. Preferably, analytes include chemical entities capable of reacting with at least one enzyme and quantitatively yielding an electrochemically reactive product that is either amperiometrically or voltammetrically detectable.

The phrases and terms “analyte measuring device,” “sensor,” and “sensor assembly” as used herein refer without limitation to an area of an analyte-monitoring device that enables the detection of at least one analyte. For example, the sensor may comprise a non-conductive portion, at least one working electrode, a reference electrode, and a counter electrode (optional), forming an electrochemically reactive surface at one location on the non-conductive portion and an electronic connection at another location on the non-conductive portion, and a one or more layers over the electrochemically reactive surface.

The phrase “capable of” as used herein, when referring to recitation of function associated with a recited structure, is inclusive of all conditions where the recited structure can actually perform the recited function. For example, the phrase “capable of” includes performance of the function under normal operating conditions, experimental conditions or laboratory conditions, as well as conditions that may not or can not occur during normal operation.

The term “cellulose acetate butyrate” as used herein refers without limitation to compounds obtained by contacting cellulose with acetic anhydride and butyric anhydride.

The term “comprising” and its grammatical equivalents, as used herein is synonymous with “including,” “containing,” or “characterized by,” and is inclusive or open-ended and does not exclude additional, unrecited elements or method steps.

The phrases “continuous analyte sensing” and “continual analyte sensing” (and the grammatical equivalents “continuously” and continually”) as used herein refer without limitation to a period of analyte concentration monitoring that is continuously, continually, and/or intermittently (but regularly) performed.

The phrase “continuous glucose sensing” as used herein refers without limitation to a period of glucose concentration monitoring that is continuously, continually, and/or intermittently (but regularly) performed. The period may, for example, at time intervals ranging from fractions of a second up to, for example, 1, 2, or 5 minutes, or longer.

The terms “crosslink” and “crosslinking” as used herein refer without limitation to joining (e.g., adjacent chains of a polymer and/or protein) by creating covalent or ionic bonds. Crosslinking may be accomplished by known techniques, for example, thermal reaction, chemical reaction or ionizing radiation (for example, electron beam radiation, UV radiation, X-ray, or gamma radiation). For example, reaction of a dialdehyde such as glutaraldehyde with a hydrophilic polymer-enzyme composition would result in chemical crosslinking of the enzyme and/or hydrophilic polymer. Crosslinking may be commensurate with sterilization of the device, for example, sterilization by e-beam or gamma irradiation.

The phrase “hydrophilic polymer-enzyme composition” refers without limitation to a physical or chemical mixture, a physical blend, a homogenous or non-homogenous mixture, a continuous or discontinuous phase, a micelle, or a dispersion of: at least one enzyme and at least one hydrophilic polymer. The hydrophilic polymer-enzyme composition may further include at least one protein, or a natural or synthetic material.

The term “break-in” as used herein refers without limitation to a time duration, after sensor deployment, where an electrical output from the sensor achieves a substantially constant value following contact of the sensor with a solution. Break-in is inclusive of configuring the sensor electronics by applying different voltage settings, starting with a higher voltage setting and then reducing the voltage setting and/or pre-treating the operating electrode with a negative electric current at a constant current density. Break-in is inclusive of chemical/electrical equilibrium of one or more of the sensor components such as membranes, layers, enzymes and electronics, and may occur prior to calibration of the sensor output. For example, following a potential input to the sensor, an immediate break-in would be a substantially constant current output from the sensor. By way of example, an immediate break-in for a glucose electrochemical sensor after contact with a solution, would be a current output representative of +/−5 mg/dL of a calibrated glucose concentration within about thirty minutes or less after deployment. The term “break-in” is well documented and is appreciated by one skilled in the art of electrochemical glucose sensors, however it may be exemplified for a glucose sensor, as the time at which reference glucose data (e.g., from an SMBG meter) is within +/−5 mg/dL of the measured glucose sensor data.

The phrase “electroactive surface” as used herein is refers without limitation to a surface of an electrode where an electrochemical reaction takes place. For example, at a predetermined potential, H₂O₂ reacts with the electroactive surface of a working electrode to produce two protons (2H+), two electrons (2e⁻) and one molecule of oxygen (O₂), for which the electrons produce a detectable electronic current. The electroactive surface may include on at least a portion thereof, a chemically or covalently bonded adhesion promoting agent, such as aminoalkylsilane, and the like.

The term “subject” as used herein refers without limitation to mammals, particularly humans and domesticated animals.

The terms “interferants,” “interferents” and “interfering species,” as used herein refer without limitation to effects and/or species that otherwise interfere with a measurement of an analyte of interest in a sensor to produce a signal that does not accurately represent the analyte measurement. For example, in an electrochemical sensor, interfering species may be compounds with oxidation potentials that substantially overlap the oxidation potential of the analyte to be measured.

The phrase “enzyme layer” as used herein refers without limitation to a permeable or semi-permeable membrane comprising one or more domains that may be permeable to reactants and/or co-reactants employed in determining the analyte of interest. As an example, an enzyme layer comprises an immobilized glucose oxidase enzyme, which catalyzes an electrochemical reaction with glucose and oxygen to permit measurement of a concentration of glucose.

The term “flux limiting membrane” as used herein refers to a semipermeable membrane that restricts or inhibits the flux of oxygen and other analytes through the semipermable membrane. Preferably, the membrane restricts or inhibits the flux of oxygen and other analytes from accessing the underlying enzyme layer. By way of example, for a glucose sensor, the membrane preferably renders oxygen in a non-rate-limiting excess. As a result, the upper limit of linearity of glucose measurement is extended to a much higher value than that which is achieved without the membrane. The phrases “flux limiting membrane” and “analyte flux limiting membrane” are used interchangeably herein.

The phrase “vinyl ester monomeric units” as used herein refers to compounds and compositions of matter which are formed from the polymerization of an unsaturated monomer having an ester functionality. For example, polypoly (ethylene-co-vinyl acetate) and copolymers thereof are compounds comprising vinyl ester monomeric units.

### Sensor System and Sensor Assembly

The aspects of the invention herein disclosed relate to the use of an analyte sensor system that measures a concentration of analyte of interest or a substance indicative of the concentration or presence of the analyte. The sensor system is a continuous device, and may be used, for example, as or part of a subcutaneous, transdermal (e.g., transcutaneous), or intravascular device. The analyte sensor may use an enzymatic, chemical, electrochemical, or combination of such methods for analyte-sensing. The output signal is typically a raw signal that is used to provide a useful value of the analyte of interest to a user, such as a patient or physician, who may be using the device. Accordingly, appropriate smoothing, calibration, and evaluation methods may be applied to the raw signal.

Generally, the sensor comprises at least a portion of the exposed electroactive surface of a working electrode surrounded by a plurality of layers. Preferably, a layer is deposited over at least a portion of the electroactive surfaces of the sensor (working electrode and optionally the reference electrode) to provide protection of the exposed electroactive surface from the biological environment and/or limit or block of interferents. An enzyme layer is deposited over at least a portion of the electroactive surface of at least one working electrode.

One exemplary embodiment described in detail below utilizes a medical device, such as a catheter, with a glucose sensor assembly. In one aspect, a medical device with an analyte sensor assembly is provided for inserting the into a subject's vascular system. The medical device with the analyte sensor assembly may include associated therewith an electronics unit associated with the sensor, and a receiver for receiving and/or processing sensor data. Although a few exemplary embodiments of continuous glucose sensors may be illustrated and described herein, it should be understood that the disclosed embodiments may be applicable to any device capable of substantially continual or substantially continuous measurement of a concentration of analyte of interest and for providing an rapid and accurate output signal that is representative of the concentration of that analyte.

### Electrode and Electroactive Surface

The electrode and/or the electroactive surface of the sensor or sensor assembly disclosed herein comprises a conductive material, such as platinum, platinum-iridium, palladium, graphite, gold, carbon, conductive polymer, alloys, ink or the like. Although the electrodes can by formed by a variety of manufacturing techniques (bulk metal processing, deposition of metal onto a substrate, or the like), it may be advantageous to form the electrodes from screen printing techniques using conductive and/or catalyzed inks or of wire. The conductive inks may be catalyzed with noble metals such as platinum and/or palladium.

In one aspect, the electrodes and/or the electroactive surfaces of the sensor or sensor assembly are formed on a flexible substrate, such as a flex circuit. In one aspect, a flex circuit is part of the sensor and comprises a substrate, conductive traces, and electrodes. The traces and electrodes may be masked and imaged onto the substrate, for example, using screen printing or ink deposition techniques. The trace and the electrodes, and the electroactive surface of the electrode may be comprised of a conductive material, such as platinum, platinum-iridium, palladium, graphite, gold, carbon, conductive polymer, alloys, ink or the like.

In one aspect, a counter electrode is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction: Glucose+O₂→Gluconate+H₂O₂. Oxidation of H₂O₂ by the working electrode is balanced by reduction of any oxygen present, or other reducible species at the counter electrode. The H₂O₂ produced from the glucose oxidase reaction reacts at the surface of working electrode and produces two protons (2H⁺), two electrons (2e⁻), and one oxygen molecule (O₂).

In one aspect, additional electrodes may be included within the sensor or sensor assembly, for example, a three-electrode system (working, reference, and counter electrodes) and/or one or more additional working electrodes configured as a baseline subtracting electrode, or which is configured for measuring additional analytes. The two working electrodes may be positioned in close proximity to each other, and in close proximity to the reference electrode. For example, a multiple electrode system may be configured wherein a first working electrode is configured to measure a first signal comprising glucose and baseline and an additional working electrode substantially similar to the first working electrode without an enzyme disposed thereon is configured to measure a baseline signal consisting of baseline only. In this way, the baseline signal generated by the additional electrode may be subtracted from the signal of the first working electrode to produce a glucose-only signal substantially free of baseline fluctuations and/or electrochemically active interfering species.

In one aspect, the sensor comprises from 2 to 4 electrodes. The electrodes may include, for example, the counter electrode (CE), working electrode (WE1), reference electrode (RE) and optionally a second working electrode (WE2). In one aspect, the sensor will have at least a CE and WE1. In one aspect, the addition of a WE2 is used, which may further improve the accuracy of the sensor measurement. In one aspect, the addition of a second counter electrode (CE2) may be used, which may further improve the accuracy of the sensor measurement.

The electroactive surface may be treated prior to application of any of the subsequent layers. Surface treatments may include for example, chemical, plasma or laser treatment of at least a portion of the electroactive surface. By way of example, the electrodes may be chemically or covalently contacted with one or more adhesion promoting agents. Adhesion promoting agents may include for example, aminoalkylalkoxylsilanes, epoxyalkylalkoxylsilanes and the like. For examples, one or more of the electrodes may be chemically or covalently contacted with a solution containing 3-glycidoxypropyltrimethoxysilane.

In some alternative embodiments, the exposed surface area of the working (and/or other) electrode may be increased by altering the cross-section of the electrode itself. Increasing the surface area of the working electrode may be advantageous in providing an increased signal responsive to the analyte concentration, which in turn may be helpful in improving the signal-to-noise ratio, for example. The cross-section of the working electrode may be defined by any regular or irregular, circular or non-circular configuration.

In some applications, cellular attack or migration of cells to the sensor can cause reduced sensitivity and/or function of the device, particularly after the first day of implantation. However, when the exposed electroactive surface of the non-working electrode is coated with a layer as herein described, reduction or elimination of local cellular contact and/or deposition of the electroactive surface is envisaged. Other methods and configurations for preventing cellular contact of the exposed electroactive surface of the non-working electrode may be used in combination with the methods disclosed herein. Therefore, in some alternative embodiments, the electroactive surface of one or more non-working electrodes (e.g., reference (RE), counter (CE), or auxiliary electrodes) may be coated with a layer capable of eliminating or reducing fouling (“anti-fouling layer”). For example, the electroactive surface of the non-working electrode may be coated with a material selected from cellulose ester derivatives, silicones, polytetrafluoroethylenes, polyethylene-co-tetrafluoroethylenes, polyolefins, polyesters, polycarbonates, biostable polytetrafluoroethylenes, homopolymers, copolymers, terpolymers of polyurethanes, polypropylenes (PP), polyvinylchloride (PVC), polyvinylidene fluoride (PVDF), polybutylene terephthalates (PBT), polymethylmethacrylates (PMMA), polyether ether ketones (PEEK), polyurethanes, cellulosic polymers, polysulfones, tetrafluoroethylene-perfluoro-3,6-dioxa-4-methyl-7-octenesulfonic acid copolymer (Nafion) and block copolymers thereof including, for example, di-block, tri-block, alternating, random and graft copolymers. Combinations of the above polymers may be used. In one preferred aspect, the anti-fouling layer is an interferant layer, such that the anti-fouling layer is effective at reducing or eliminating diffusion of interfering species relative to, for example, hydrogen peroxide.

In one aspect, the anti-fouling layer is formed from one or more cellulosic derivatives. Cellulosic derivatives can include, but are not limited to, cellulose esters and cellulose ethers. In general, cellulosic derivatives include polymers such as cellulose acetate, cellulose acetate butyrate, 2-hydroxyethyl cellulose, cellulose acetate phthalate, cellulose acetate propionate, cellulose acetate trimellitate, and the like, as well as their copolymers and terpolymers with other cellulosic or non-cellulosic monomers. While cellulosic derivatives are generally preferred, other polymeric polysaccharides having similar properties to cellulosic derivatives may also be employed.

In one aspect, the anti-fouling layer deposited on the electroactive surface of the at least one non-working electrode is formed from cellulose acetate butyrate. Cellulose acetate butyrate is a cellulosic polymer having both acetyl and butyl groups, and may also include hydroxyl groups. A cellulose acetate butyrate having about 35% or less acetyl groups, about 10% to about 25% butyryl groups, and hydroxyl groups making up the remainder may be used. A cellulose acetate butyrate having from about 25% to about 34% acetyl groups and from about 15 to about 20% butyryl groups may also be used, however, other amounts of acetyl and butyryl groups may be used. A preferred cellulose acetate butyrate contains from about 28% to about 30% acetyl groups and from about 16 to about 18% butyryl groups.

In one aspect, the anti-fouling layer may be a vinyl polymer appropriate for use in sensor devices. Examples of materials which may be used to make anti-fouling layers include vinyl polymers having vinyl acetate monomeric units. In a preferred embodiment, the anti-fouling layer comprises poly ethylene vinylacetate (EVA polymer) having a vinyl acetate content of less than 33 wt. %. In other aspects, the anti-fouling layer comprises poly ethylene vinylacetate (EVA polymer) having a vinyl acetate content of about 25 wt. %. In a preferred aspect, the anti-fouling layer comprises poly ethylene vinylacetate (EVA polymer) having a vinyl acetate content of about 18 wt. %.

Thus, in various aspects of the sensor disclosed and described herein, the sensor further comprises a reference electrode, a counter electrode, an auxiliary electrode, or combinations thereof, having disposed thereon, at least one anti-fouling layer.

At Least One Interferent-Reducing layer

Interferents may be molecules or other species that may be reduced or oxidized at the electrochemically reactive surfaces of the sensor, either directly or via an electron transfer agent, to produce a false positive analyte signal (e.g., a non-analyte-related signal). This false positive signal generally causes the subject's analyte concentration to appear higher than the true analyte concentration. For example, in a hypoglycemic situation, where the subject has ingested an interferent (e.g., acetaminophen), the artificially high glucose signal may lead the subject or health care provider to believe that they are euglycemic or, in some cases, hyperglycemic. As a result, the subject or health care provider may make inappropriate or incorrect treatment decisions. In other events, significant amounts of electrochemical species generated in the enzyme layer may be lost by diffusion or transport away from the electrochemical surface.

In one aspect, the at least one interferent-reducing layer comprising vinyl ester monomeric units is provided on the sensor or sensor assembly that substantially reduces or restricts the passage there through of one or more interfering species. Interfering species for a glucose sensor include, for example, acetaminophen, ascorbic acid, bilirubin, cholesterol, creatinine, dopamine, ephedrine, ibuprofen, L-dopa, methyl dopa, salicylate, tetracycline, tolazamide, tolbutamide, triglycerides, urea and uric acid. The interferent-reducing layer may be less permeable to one or more of the interfering species than to a target analyte species, such as hydrogen peroxide. In another aspect, the at least one interferent-reducing layer comprising vinyl ester monomeric units is provided on the sensor or sensor assembly that substantially reduces or restricts diffusion or transport of hydrogen peroxide, for example, from the enzyme layer. Thus, when the at least one interferent-reducing layer comprising vinyl ester monomeric units covers the enzyme layer over the electroactive surface, the hydrogen peroxide generated will be more likely to be detected.

In one aspect, the at least one interferent-reducing layer comprising vinyl ester monomeric units is a combination with one or more materials selected from cellulose ester derivatives, silicones, polytetrafluoroethylene, polyethylene-co-tetrafluoroethylene, polyolefin, polyester, polycarbonate, biostable polytetrafluoroethylene, homopolymers, copolymers, terpolymers or blends of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones, tetrafluoroethylene-perfluoro-3,6-dioxa-4-methyl-7-octenesulfonic acid copolymer (Nafion) and block copolymers thereof including, for example, di-block, tri-block, alternating, random and graft copolymers. Blends of the above polymers may be used.

In an embodiment, the at least one interferent-reducing layer comprising vinyl ester monomeric units is a combination of and one or more cellulosic derivatives. In one aspect, mixed ester cellulosic derivatives may be used, for example, cellulose acetate butyrate, cellulose acetate phthalate, cellulose acetate propionate, cellulose acetate trimellitate, as well as their copolymers and terpolymers, with other cellulosic or non-cellulosic monomers, including cross-linked variations of the above. Other polymers, such as polymeric polysaccharides having similar properties to cellulosic derivatives, may be used as an interference material or in combination with the above cellulosic derivatives. Other esters of cellulose may be blended with the mixed ester cellulosic derivatives.

In one aspect, the combination of a polymer comprising vinyl ester monomeric units and cellulose acetate butyrate is used. Cellulose acetate butyrate is a cellulosic polymer having both acetyl and butyl groups, and hydroxyl groups. A cellulose acetate butyrate having about 35% or less acetyl groups, about 10% to about 25% butyryl groups, and hydroxyl groups making up the remainder may be used. A cellulose acetate butyrate having from about 25% to about 34% acetyl groups and from about 15 to about 20% butyryl groups may also be used, however, other amounts of acetyl and butyryl groups may be used. A preferred cellulose acetate butyrate contains from about 28% to about 30% acetyl groups and from about 16 to about 18% butyryl groups.

Cellulose acetate butyrate with a molecular weight of about 10,000 daltons to about 75,000 daltons is preferred, preferably from about 15,000, 20,000, or 25,000 daltons to about 50,000, 55,000, 60,000, 65,000, or 70,000 daltons, and more preferably about 65,000 daltons is employed. In certain embodiments, however, higher or lower molecular weights may be used or a blend of two or more cellulose acetate butyrates having different molecular weights may be used.

A plurality of layers of polymer comprising vinyl ester monomeric units and cellulose acetate butyrate may be constructed to form the at least one interferent-reducing layer in some embodiments, for example, two or more layers may be employed. It may be desirable to employ a mixture of cellulose acetate butyrates with different molecular weights in a single solution, or to deposit multiple layers of cellulose acetate butyrate from different solutions comprising cellulose acetate butyrate of different molecular weights, different concentrations, and/or different chemistries (e.g., wt. % functional groups), or to sandwich the polymer comprising vinyl ester monomeric units between cellulose derivatives. Additional substances in the casting solutions or dispersions may be used, e.g., casting aids, defoamers, surface tension modifiers, functionalizing agents, crosslinking agents, other polymeric substances, substances capable of modifying the hydrophilicity/hydrophobicity of the resulting layer, and the like.

The at least one interferent-reducing layer comprising vinyl ester monomeric units may be sprayed, cast, coated, or dipped directly to the electroactive surface(s) of the sensor. The dispensing of the interference material may be performed using any known thin film technique. Two, three or more layers of interference material may be formed by the sequential application and curing and/or drying of the casting solution.

The concentration of solids in the casting solution may be adjusted to deposit a sufficient amount of solids or film on the electrode in one layer (e.g., in one dip or spray) to form a layer sufficient to block an interferant with an oxidation or reduction potential otherwise overlapping that of a measured species (e.g., H₂O₂), measured by the sensor. For example, the casting solution's percentage of solids may be adjusted such that only a single layer is required to deposit a sufficient amount to form a functional interference layer that substantially prevents or reduces the equivalent glucose signal of the interferant measured by the sensor. A sufficient amount of interference material would be an amount that substantially prevents or reduces the equivalent glucose signal of the interferant of less than about 30, 20 or 10 mg/dl. By way of example, at least one of the interferent-reducing layers is preferably configured to substantially block about 30 mg/dl of an equivalent glucose signal response that otherwise would be produced by acetaminophen by a sensor without an interference layer. Such equivalent glucose signal response produced by acetaminophen would include a therapeutic dose of acetaminophen. Any number of coatings or layers formed in any order may be suitable for forming the layer of the embodiments disclosed herein.

In one aspect, the at least one interferent-reducing layer comprising vinyl ester monomeric units is deposited either directly onto an electroactive surfaces of the sensor or onto a material or layer (e.g., electrode layer, enzyme layer) in direct contact with the surface of the electrode.

In another aspect, the at least one interferent-reducing layer comprising vinyl ester monomeric units is deposited either directly onto a material or layer (e.g., electrode layer, enzyme layer) that is on or in direct contact with an electroactive surface of the sensor electrode.

In yet another aspect, at least two interferent-reducing layer comprising vinyl ester monomeric units have positioned between them a material or layer (e.g., electrode layer, enzyme layer).

The at least one interferent-reducing layer comprising vinyl ester monomeric units may be applied to provide a thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 1, 1.5 or 2 microns to about 2.5 or 3 microns. Thicker membranes may also be desirable in certain embodiments, but thinner membranes may be generally preferred because they generally have a lower effect on the rate of diffusion of hydrogen peroxide from the enzyme membrane to the electrodes.

### Enzyme Layer

The sensor or sensor assembly disclosed herein includes an enzyme layer for interacting with the analyte and/or co-analyte of interest. In one aspect, the enzyme layer comprises at least one separate enzyme-containing layer or is a layer comprising a mixture of a hydrophilic polymer and an enzyme . The hydrophilic polymer functions as an electrode layer whether it is deposited separately or in combination with the enzyme containing layer. In one aspect, the enzyme layer comprises an enzyme layer or hydrophilic polymer-enzyme composition deposited directly onto at least a portion of the electroactive surface. In another aspect, the enzyme layer comprises an enzyme layer or hydrophilic polymer-enzyme composition deposited directly onto at least a portion of the at least one interferent-reducing layer.

In one aspect, the enzyme layer comprises a enzyme and a hydrophilic polymer selected from poly-N-vinylpyrrolidone (PVP), poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polymers with pendent ionizable groups and copolymers thereof. In one aspect, the enzyme layer comprises glucose oxidase, poly-N-vinylpyrrolidone and an amount of crosslinking agent sufficient to immobilize the enzyme.

The molecular weight of the hydrophilic polymer of the enzyme layer is such that fugitive species are prevented or substantially inhibited from leaving the sensor environment and more particularly, fugitive species are prevented or substantially inhibited from leaving the enzyme's environment when the sensor is initially deployed.

The hydrophilic polymer-enzyme composition of the enzyme layer may further include at least one protein and/or natural or synthetic material. For example, the hydrophilic polymer-enzyme composition of the enzyme layer may further include, for example, serum albumins, polyallylamines, polyamines and the like, as well as combination thereof.

In one aspect, the enzyme is immobilized in the sensor. The enzyme may be encapsulated within the hydrophilic polymer and may be cross-linked or otherwise immobilized therein. The enzyme may be cross-linked or otherwise immobilized optionally together with at least one protein and/or natural or synthetic material. In one aspect, the hydrophilic polymer-enzyme composition comprises glucose oxidase, bovine serum albumin, and poly-N-vinylpyrrolidone. The composition may further include a cross-linking agent, for example, a dialdehyde such as glutaraldehyde, to cross-link or otherwise immobilize the components of the composition.

In one aspect, other proteins or natural or synthetic materials may be substantially excluded from the hydrophilic polymer-enzyme composition of the enzyme layer. For example, the hydrophilic polymer-enzyme composition may be substantially free of bovine serum albumin Bovine albumin-free compositions may be desirable for meeting various governmental regulatory requirements. Thus, in one aspect, the enzyme layer comprises glucose oxidase and a sufficient amount of cross-linking agent, for example, a dialdehyde such as glutaraldehyde, to cross-link or otherwise immobilize the enzyme. In other aspect, the enzyme layer comprises glucose oxidase, poly-N-vinylpyrrolidone and a sufficient amount of cross-linking agent to cross-link or otherwise immobilize the enzyme.

The enzyme layer thickness may be from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. Preferably, the enzyme layer is deposited by spray or dip coating, however, other methods of forming the enzyme layer may be used. The enzyme layer may be formed by dip coating and/or spray coating one or more layers at a predetermined concentration of the coating solution, insertion rate, dwell time, withdrawal rate, and/or desired thickness.

### Analyte Flux Limiting Membrane

In one aspect, an analyte flux limiting membrane (or flux limiting membrane) is positioned over the subsequent layers described above, where the analyte flux limiting membrane alters or changes the rate of flux of one or more of the analytes or co-analytes of interest, for example, glucose and oxygen. Although the following description is directed to a flux limiting membrane for an electrochemical glucose sensor, the flux limiting membrane may be modified for other analytes and co-analytes as well.

In one aspect, the flux limiting membrane comprises a semi-permeable material that controls the flux of oxygen and glucose to the underlying layers, preferably providing oxygen in a non-rate-limiting excess. As a result, the upper limit of linearity of glucose measurement is extended to a much higher value than that which is achieved without the flux limiting membrane. In one embodiment, the flux limiting membrane exhibits an oxygen to glucose permeability ratio of from about 50:1 or less to about 400:1 or more, preferably about 200:1. Other flux limiting membranes may be used or combined, such as a membrane with both hydrophilic and hydrophobic polymeric regions, to control the diffusion of analyte and optionally co-analyte to an analyte sensor. For example, a suitable membrane may include a hydrophobic polymer matrix component such as a polyurethane, or polyetherurethaneurea. The flux limiting membrane may function in some extent, as an interferent layer, such as the combination of hydrophobic silicone polyurethane, such as Carbosil, with a hydrophilic polymer, such as polyvinylpyrrolidone. In one aspect, the material that forms the basis of the hydrophobic matrix of the layer can be any of those known in the art as appropriate for use as membranes in sensor devices and as having sufficient permeability to allow relevant compounds to pass through it, for example, to allow an oxygen molecule to pass through the layer from the sample under examination in order to reach the active enzyme or electrochemical electrodes. For example, non-polyurethane type layers such as vinyl polymers, polyethers, polyesters, polyamides, or thin-film, track-etched polycarbonates, inorganic polymers such as polysiloxanes and polycarbosiloxanes, natural polymers such as cellulosic and protein based materials, and mixtures or combinations thereof may be used.

In one aspect, the analyte flux limiting membrane comprises a polyethylene oxide component. For example, a hydrophobic-hydrophilic copolymer comprising polyethylene oxide is a polyurethane polymer that includes about 20% hydrophilic polyethylene oxide. The polyethylene oxide portions of the copolymer are thermodynamically driven to separate from the hydrophobic portions (e.g., the urethane portions) of the copolymer and the hydrophobic polymer component. The 20% polyethylene oxide-based soft segment portion of the copolymer used to form the final blend affects the water pick-up and subsequent glucose permeability of the membrane.

In one aspect, the membrane comprises a semi-permeable material that controls the flux of oxygen and glucose to the underlying enzyme layer, preferably providing oxygen in a non-rate-limiting excess. As a result, the upper limit of linearity of glucose measurement is extended to a much higher value than that which is achieved without the membrane. In one embodiment, the membrane exhibits an oxygen to glucose permeability ratio of from about 50:1 or less to about 400:1 or more, preferably about 200:1.

The material that comprises the membrane may be a vinyl polymer appropriate for use in sensor devices having sufficient permeability to allow relevant compounds to pass through it, for example, to allow an oxygen molecule to pass through in order to reach the active enzyme or electrochemical electrodes. Examples of materials which may be used to make the membrane include vinyl polymers having vinyl ester monomeric units. In a preferred embodiment, a flux limiting membrane comprises poly ethylene vinyl acetate (EVA polymer). In other aspects, the flux limiting membrane comprises poly(methylmethacrylate-co-butyl methacrylate) blended with the EVA polymer. The EVA polymer or its blends may be cross-linked, for example, with diglycidyl ether. Films of EVA are very elastomeric, which may provide resiliency to the sensor for navigating a tortuous path, for example, into venous anatomy. In one aspect, the flux limiting membrane and the at least one interferent-reducing layer comprising vinyl ester monomeric units are both EVA polymers, albeit, of different % vinyl acetate content.

The EVA polymer may be provided from a source having a composition of about 40 wt. % vinyl acetate (EVA-40). The EVA polymer is preferably dissolved in a solvent for dispensing on the sensor or sensor assembly. The solvent should be chosen for its ability to dissolve EVA polymer, to promote adhesion to the sensor substrate and enzyme electrode, and to form a solution that may be effectively applied (e.g. spray-coated or dip coated). Solvents such as cyclohexanone, paraxylene, and tetrahydrofuran may be suitable for this purpose. The solution may include about 0.5 wt. % to about 6.0 wt. % of the EVA polymer. In addition, the solvent should be sufficiently volatile to evaporate without undue agitation to prevent issues with the underlying enzyme, but not so volatile as to create problems with the spray process. In a preferred embodiment, the vinyl acetate component of the flux limiting membrane includes about 20% vinyl acetate. In preferred embodiments, the flux limiting membrane is deposited onto the enzyme layer to yield a layer thickness of from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 5, 5.5 or 6 microns to about 6.5, 7, 7.5 or 8 microns. The flux limiting membrane may be deposited onto the enzyme layer by spray coating or dip coating. In one aspect, the flux limiting membrane is deposited on the enzyme layer by dip coating a solution of from about 1 wt. % to about 10 wt. % EVA polymer and from about 95 wt. % to about 99 wt. % solvent.

In one aspect of the present disclosure, an electrochemical analyte sensor is provided comprising an EVA flux limiting membrane of a defined composition covering the enzyme layer, and an EVA interferent-reducing layer of a different composition than the flux limiting layer, and at least a portion of the electroactive surface.

### Bioactive Agents

In some alternative embodiments, a bioactive agent may be optionally incorporated into the above described sensor system, such that the bioactive diffuses out into the biological environment adjacent to the sensor. Additionally or alternately, a bioactive agent may be administered locally at the exit-site or implantation-site. Suitable bioactive agents include those that modify the subject's tissue response to any of the sensor or components thereof. For example, bioactive agents may be selected from anti-inflammatory agents, anti-infective agents, anesthetics, inflammatory agents, growth factors, immunosuppressive agents, antiplatelet agents, anti-coagulants, anti-proliferates, ACE inhibitors, cytotoxic agents, anti-barrier cell compounds, vascularization-inducing compounds, anti-sense molecules, or mixtures thereof. In one aspect, heparin or a heparin derivative is applied to the outermost surface of the sensor.

### Flexible Substrate Sensor Assembly Adapted for Intravenous Insertion

In one aspect, an electrochemical analyte sensor assembly may be configured for an intravenous insertion to a vascular system of a subject. In order to accommodate the sensor within the confined space of a device suitable for intravenous insertion, the sensor assembly may comprise a flexible substrate, such as a flex circuit. For example, the flexible substrate of the flex circuit may be configured as a thin conductive electrodes coated on a non-conductive material such as a thermoplastic or thermoset. Conductive traces may be formed on the non-conductive material and electrically coupled to the thin conductive electrodes. The electrodes of the flex circuit may be as described above.

The flex circuit may comprise at least one reference electrode and at least one working electrode, the at least one working electrode having an electroactive surface capable of providing a detectable electrical output upon interaction with an electrochemically detectable species. The flex circuit may further comprise at least one counter electrode. In one aspect, the flex circuit contains two or more working electrodes and two or more counter electrodes. In one aspect, the flex circuit contains two or more working electrodes, two or more blank electrodes and two or more counter electrodes.

In one aspect, the at least one interferent-reducing layer comprising vinyl ester monomeric units is placed in direct contact with and at least partially covering a portion of the electroactive surface of the working electrode of the flex circuit. An enzyme layer comprising a hydrophilic polymer-enzyme composition capable of enzymatically interacting with an analyte so as to provide the electrochemically detectable species, is placed such that at least a portion thereof is in direct contact with and at least partially covering the at least one interferent-reducing layer comprising vinyl ester monomeric units. A flux limiting membrane, such as a membrane that alters the flux of an analyte of interest, may be placed such that it covers the hydrophilic polymeric layer, the at least one interferent-reducing layer comprising vinyl ester monomeric units, and at least a portion of the electroactive surface of the flex circuit.

In another aspect, an enzyme layer comprising a hydrophilic polymer-enzyme composition capable of enzymatically interacting with an analyte so as to provide the electrochemically detectable species, is placed such that at least a portion thereof is in direct contact with the electroactive surface of the working electrode of the flex circuit. The at least one interferent-reducing layer comprising vinyl ester monomeric units is placed in direct contact with and at least partially covering a portion of the enzyme layer. A flux limiting membrane, such as a membrane that alters the flux of an analyte of interest, may be placed such that it covers the hydrophilic polymeric layer, the at least one interferent-reducing layer comprising vinyl ester monomeric units, and at least a portion of the electroactive surface of the flex circuit.

The flex circuit preferably is configured to be electrically configurable to a control unit. An example of an electrode of a flex circuit and it construction is found in co-assigned U.S. Application Nos. 2007/0202672 and 2007/0200254, incorporated herein by reference in their entirety.

Medical devices adaptable to the sensor assembly as described above include, but are not limited to a central venous catheter (CVC), a pulmonary artery catheter (PAC), a probe for insertion through a CVC or PAC or through a peripheral IV catheter, a peripherally inserted catheter (PICC), Swan-Ganz catheter, an introducer or an attachment to a Venous Arterial blood Management Protection (VAMP) system. Any size/type of Central Venous Catheter (CVC) or intravenous devices may be used or adapted for use with the sensor assembly.

For the foregoing discussion, the implementation of the sensor or sensor assembly is disclosed as being placed within a catheter, however, other devices as described above are envisaged and incorporated in aspects of the invention. The sensor assembly will preferably be applied to the catheter so as to be flush with the OD of the catheter tubing. This may be accomplished, for example, by thermally deforming the OD of the tubing to provide a recess for the sensor. The sensor assembly may be bonded in place, and sealed with an adhesive (ie. urethane, 2-part epoxy, acrylic, etc.) that will resist bending/peeling, and adhere to the urethane CVC tubing, as well as the materials of the sensor. Small diameter electrical wires may be attached to the sensor assembly by soldering, resistance welding, or conductive epoxy. These wires may travel from the proximal end of the sensor, through one of the catheter lumens, and then to the proximal end of the catheter. At this point, the wires may be soldered to an electrical connector.

The sensor assembly as disclosed herein can be added to a catheter in a variety of ways. For example, an opening may be provided in the catheter body and a sensor or sensor assembly may be mounted inside the lumen at the opening so that the sensor would have direct blood contact. In one aspect, the sensor or sensor assembly may be positioned proximal to all the infusion ports of the catheter. In this configuration, the sensor would be prevented from or minimized in measuring otherwise detectable infusate concentration instead of the blood concentration of the analyte. Another aspect, an attachment method may be an indentation on the outside of the catheter body and to secure the sensor inside the indentation. This may have the added advantage of partially isolating the sensor from the temperature effects of any added infusate. Each end of the recess may have a skived opening to 1) secure the distal end of the sensor and 2) allow the lumen to carry the sensor wires to the connector at the proximal end of the catheter.

Preferably, the location of the sensor assembly in the catheter will be proximal (upstream) of any infusion ports to prevent or minimize IV solutions from affecting analyte measurements. In one aspect, the sensor assembly may be about 2.0 mm or more proximal to any of the infusion ports of the catheter.

In another aspect, the sensor assembly may be configured such that flushing of the catheter (i.e., saline solution) may be employed in order to allow the sensor assembly to be cleared of any material that may interfere with its function.

### Sterilization of the Sensor or Sensor Assembly

Generally, the sensor or the sensor assembly as well as the device that the sensor is adapted to are sterilized before use, for example, in a subject. Sterilization may be achieved using radiation (e.g., electron beam or gamma radiation), ethylene oxide or flash-UV sterilization, or other means know in the art.

Disposable portions, if any, of the sensor, sensor assembly or devices adapted to receive and contain the sensor preferably will be sterilized, for example using e-beam or gamma radiation or other know methods. The fully assembled device or any of the disposable components may be packaged inside a sealed non-breathable container or pouch.

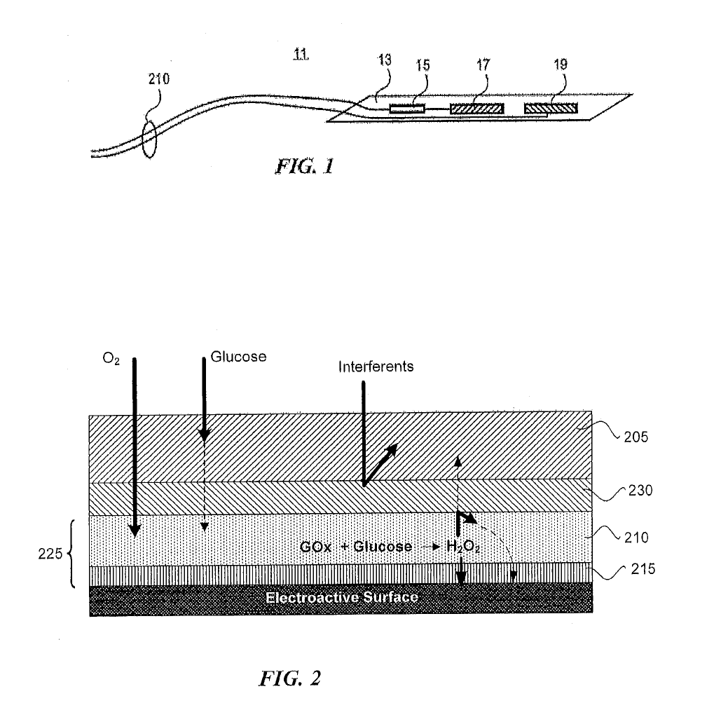

Referring now to the Figures, FIG. 1 is an amperometric sensor 11 in the form of a flex circuit that incorporates a sensor embodiment disclosed herein. The sensor or sensor 11 may be formed on a substrate 13 (e.g., a flex substrate, such as copper foil laminated with polyimide). One or more electrodes 15, 17 and 19 may be attached or bonded to a surface of the substrate 13. The sensor 11 is shown with a reference electrode 15, a counter electrode 17, and a working electrode 19. In another embodiment, one or more additional working electrodes may be included on the substrate 13. Electrical wires 210 may transmit power to the electrodes for sustaining an oxidation or reduction reaction, and may also carry signal currents to a detection circuit (not shown) indicative of a parameter being measured. The parameter being measured may be any analyte of interest that occurs in, or may be derived from, blood chemistry. In one embodiment, the analyte of interest may be hydrogen peroxide, formed from reaction of glucose with glucose oxidase, thus having a concentration that is proportional to blood glucose concentration.

FIG. 2 depicts a cross-sectional side view of a portion of a sensor aspect herein disclosed. Enzyme layer 210 that is selected to chemically react when the sensor is exposed to certain reactants, for example, found in the bloodstream, and optional electrode layer 215 are at least partially coated distally from the electroactive surface of a working electrode. For example, in an embodiment for a glucose sensor, enzyme layer 210 may contain glucose oxidase, such as may be derived from Aspergillus niger (EC 1.1.3.4), type II or type VII. Enzyme layer 210 and electrode layer 215 may be a homogenous or inhomogeneous mixture of layers, such as indicated by combined layer 225. Interferent-reducing layer 230 comprising vinyl ester monomeric units is coated more distally from the electroactive surface than enzyme layer 210. Interferent-reducing layer 230 comprising vinyl ester monomeric units at least partially covers the enzyme layer and functions to block interferents as well as reduce diffusion of hydrogen peroxide from the enzyme layer. Flux limiting membrane 205 covers enzyme layer 210 and interferent-reducing layer 230 comprising vinyl ester monomeric units and at least a portion of electroactive surface. Flux limiting membrane 205 may selectively allow diffusion, from blood to the enzyme layer 210, a blood component that reacts with the enzyme. In a glucose sensor embodiment, the flux limiting membrane 205 passes an abundance of oxygen, and selectively limits glucose, to the enzyme layer 210. In addition, a flux limiting membrane 205 that has adhesive properties may mechanically seal the enzyme layer 210 to the sub-layers and/or working electrode, and may also seal the working electrode to the sensor substrate. A flux limiting membrane formed from an EVA polymer may serve as a flux limiter at the top of the electrode, but also serve as a sealant or encapsulant at the enzyme/electrode boundary and at the electrode/substrate boundary. An additional biocompatible layer (not shown), including a biocompatible anti-thrombotic substance such as heparin or heparin derivative, may be added onto the flux limiting membrane 205.

FIG. 3 shows a cross sectional side view of another aspect of the sensor disclosed herein. Thus, interferent-reducing layer 235 comprising vinyl ester monomeric units is coated distally from the electroactive surface and functions to block interferents. In this aspect, interferent-reducing layer 235 comprising vinyl ester monomeric units permits passage of hydrogen peroxide from the enzyme layer to the electroactive surface. Enzyme layer 210 is positioned more distally from the electroactive surface than 235 comprising vinyl ester monomeric units. As shown, a separate electrode layer 215 at least partially covers the electroactive surface of the working electrode. Flux limiting membrane 205 covers enzyme layer 210 and interferent-reducing layer 235 and at least a portion of the electrode.

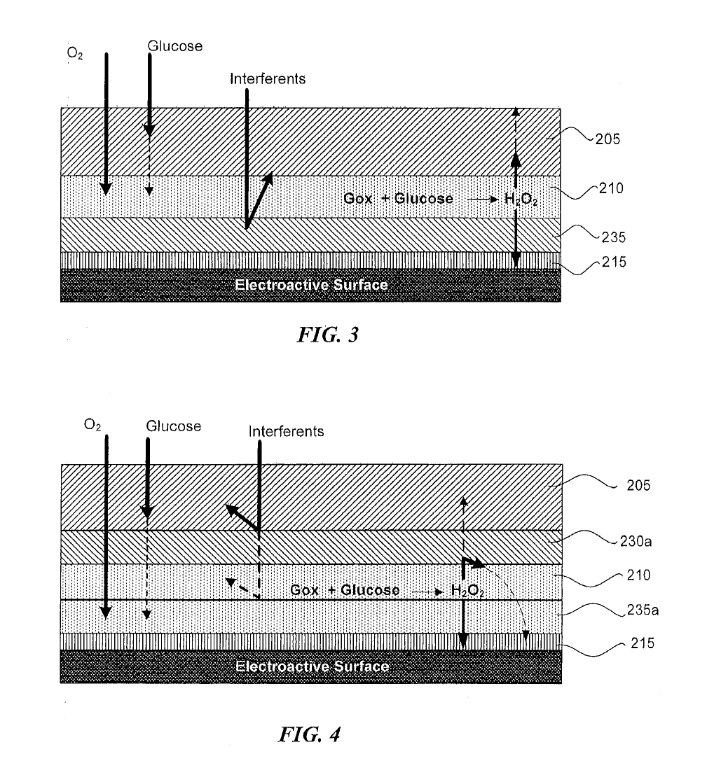

FIG. 4 shows a cross sectional side view of another aspect of the sensor disclosed herein. In this aspect, the enzyme layer is sandwiched between first and second polymer layers, at least one of the first and second polymer layers comprising vinyl ester monomeric units. The first polymer layer 235a blocking interferents from reaching the electroactive surface, the second polymer layer 230a reducing hydrogen peroxide diffusion from the enzyme layer. Thus, first polymer layer 235a is coated distally from the electroactive surface and functions to block interferents. In this aspect, first polymer layer 235a permits passage of hydrogen peroxide from the enzyme layer to the electroactive surface. Enzyme layer 210 is positioned more distally from the electroactive surface than interferent layer 235. As shown, a separate electrode layer 215 at least partially covers the electroactive surface of the working electrode. Second polymer layer 230a is coated more distally from the electroactive surface than enzyme layer 210. Second polymer layer 230a at least partially covers the enzyme layer and functions to block interferents as well as reduce diffusion of hydrogen peroxide from the enzyme layer. Flux limiting membrane 205 covers enzyme layer 210 and first and second polymer layers 230a, 235a and at least a portion of the electrode.

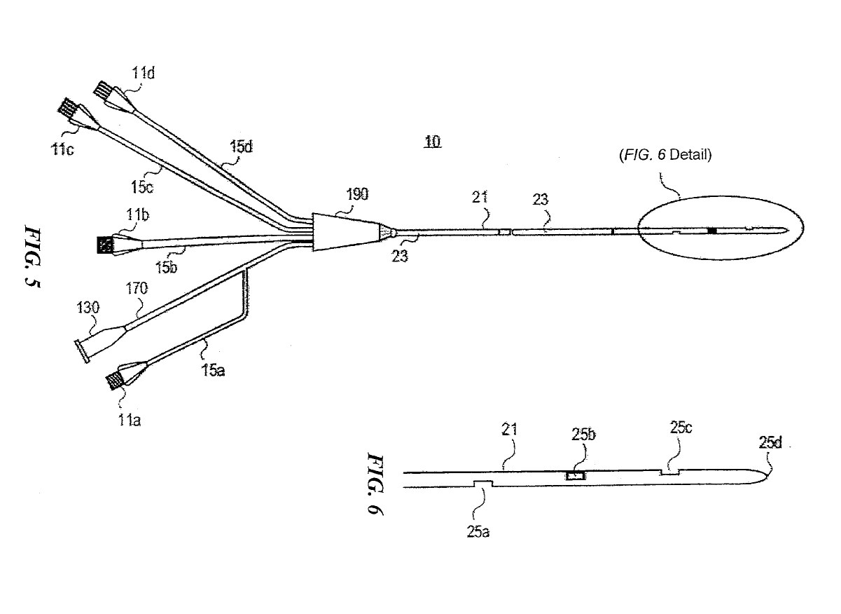

Referring now to FIGS. 5-6, aspects of the sensor adapted to a central line catheter with a sensor or sensor assembly are discussed as exemplary embodiments, without limitation to any particular intravenous device. FIG. 5 shows a sensor assembly within a multilumen catheter. The catheter assembly 10 may include multiple infusion ports 11a, 11b, 11c, 11d and one or more electrical connectors 130 at its most proximal end. A lumen 15a, 15b, 15c or 15d may connect each infusion port 11a, 11b, 11c, or 11d, respectively, to a junction 190. Similarly, the conduit 170 may connect an electrical connector 130 to the junction 190, and may terminate at junction 190, or at one of the lumens 15a-15d (as shown). Although the particular embodiment shown in FIG. 5 is a multi-lumen catheter having four lumens and one electrical connector, other embodiments having other combinations of lumens and connectors are possible, including a single lumen catheter, a catheter having multiple electrical connectors, etc. In another embodiment, one of the lumens and the electrical connector may be reserved for a probe or other sensor mounting device, or one of the lumens may be open at its proximal end and designated for insertion of the probe or sensor mounting device.

The distal end of the catheter assembly 10 is shown in greater detail in FIG. 6. At one or more intermediate locations along the distal end, the tube 21 may define one or more ports formed through its outer wall. These may include the intermediate ports 25a, 25b, and 25c, and an end port 25d that may be formed at the distal tip of tube 21. Each port 25a-25d may correspond respectively to one of the lumens 15a-15d. That is, each lumen may define an independent channel extending from one of the infusion ports 11a-11d to one of the tube ports 25a-25d. The sensor assembly may be presented to the sensing environment via positioning at one or more of the ports to provide contact with the medium to be analyzed.

Central line catheters may be known in the art and typically used in the Intensive Care Unit (ICU)/Emergency Room of a hospital to deliver medications through one or more lumens of the catheter to the patient (different lumens for different medications). A central line catheter is typically connected to an infusion device (e.g. infusion pump, IV drip, or syringe port) on one end and the other end inserted in one of the main arteries or veins near the patient's heart to deliver the medications. The infusion device delivers medications, such as, but not limited to, saline, drugs, vitamins, medication, proteins, peptides, insulin, neural transmitters, or the like, as needed to the patient. In alternative embodiments, the central line catheter may be used in any body space or vessel such as intraperitoneal areas, lymph glands, the subcutaneous space, the lungs, the digestive tract, or the like and may determine the analyte or therapy in body fluids other than blood. The central line catheter may be a double lumen catheter. In one aspect, an analyte sensor is built into one lumen of a central line catheter and is used for determining characteristic levels in the blood and/or bodily fluids of the user. However, it will be recognized that further embodiments may be used to determine the levels of other agents, characteristics or compositions, such as hormones, cholesterol, medications, concentrations, viral loads (e.g., HIV), or the like. Therefore, although aspects disclosed herein may be primarily described in the context of glucose sensors used in the treatment of diabetes/diabetic symptoms, the aspects disclosed may be applicable to a wide variety of patient treatment programs where a physiological characteristic is monitored in an ICU, including but not limited to blood gases, pH, temperature and other analytes of interest in the vascular system.

In another aspect, a method of intravenously measuring an analyte in a subject is provided. The method comprises providing a catheter comprising the sensor assembly as described herein and introducing the catheter into the vascular system of a subject. The method further comprises measuring an analyte.

### EXAMPLES

Preparation of sensors—Test Board UNF: Poly(4-styrenesulfonic acid-co-maleic acid), sodium salt, 25 wt % solution in water (Aldrich) was diluted to 0.25 wt. % using deionized water. This solution was applied to all working and blank electrodes of a flex circuit sensor as described in U.S. Patent Application No. 2007/0200254, using an electronic fluid dispenser (EFD). After dispense, the test board was placed in a 60° C. oven for about 5 minutes to dry the solution. Subsequently, a 1 wt % of poly (ethylene-co-vinyl acetate) 25 wt % vinyl acetate (Aldrich) in p-xylene, heated between about 40° C. to about 50° C., was applied by hand using a swab tip applicator and dried at about 60° C. for about 20 minutes. This heated solution was applied to the working and blank electrodes previously coated with the poly(4-styrenesulfonic acid-co-maleic acid), sodium salt, 25 wt % solution on some, but not all, of the sensors on the UNF test board.

To some of the remaining working and blank electrodes previously coated with the poly(4-styrenesulfonic acid-co-maleic acid), sodium salt, 25 wt % solution, a 1 wt % of poly (ethylene-co-vinyl acetate) 40 wt % vinyl acetate (Aldrich) in p-xylene (at room temperature) was applied by hand using a swab tip applicator. After application of the poly (ethylene-co-vinyl acetate) p-xylene solution, the test board was placed in a 60° C. oven for about 20 minutes to dry.

A solution containing 5 wt % glucose oxidase, glutaraldehyde and bovine serum albumin was than deposited on all working and blank electrodes and dried at room temperature for about 2 hours.

The entire test board was dip coated in a 9 wt % poly (ethylene-co-vinyl acetate) 40 wt % vinyl acetate (Aldrich) in p-xylene at room temperature. The dip coating conditions were 2 minutes dwell time and 500 mm/minute withdrawal rate. After dip coating, the entire test board was placed in a 60° C. oven for about 15 minutes to dry.

Test Board V0051: Poly(4-styrenesulfonic acid-co-maleic acid), sodium salt, 25 wt % solution in water (Aldrich) was diluted to 0.25 wt. % using deionized water. This solution was applied to all working and blank electrodes of a flex circuit as described above, using an electronic fluid dispenser (EFD). After dispense, the test board was placed in a 60° C. oven for about 5 minutes to dry. Subsequently, a 1.5 wt % of poly (ethylene-co-vinyl acetate) 12 wt % vinyl acetate (Aldrich) in p-xylene heated to 60° C. was applied by dip coating. The dip coating conditions were 2 minute dwell time and 500 mm/min withdrawal rate. Subsequently, the test board V0051 was placed in a 60° C. oven for about 25 minutes to dry.

A solution containing 5 wt % glucose oxidase, glutaraldehyde and bovine serum albumin was than deposited on all working and blank electrodes. After application of the glucose oxidase solution the test board remained at room temperature for about 30 minutes to dry.

The entire test board was dip coated in a 9 wt % poly (ethylene-co-vinyl acetate) 40 wt % vinyl acetate (Aldrich) in p-xylene heated to about 30° C. The dip coating conditions were 2 minutes dwell time and 500 mm/minute withdrawal rate. After dip coating the entire test board was placed in a 60° C. oven for about 15 minutes to dry.

### Characterization of Glucose Sensitivity:

Sensors were sequentially immersed into a series of glucose solutions of glucose concentrations of 100, 200 and 400 mg/dL, respectively. The sensors were connected to potentiostats and the output signal recorded. Plots of sensor current as a function of glucose concentration yielded a glucose sensitivity slope (current/mg⁻¹dL⁻¹) for each sensor.

Characterization of Acetaminophen Sensitivity: Sensors prepared as described above were placed in a 2 mg/dL solution of acetaminophen. The sensors were connected to potentiostat and sensor current was recorded. This acetaminophen sensor current was converted into an equivalent glucose signal by dividing this current by the glucose slope sensitivity as described above. The data for each sensor is summarized in the TABLE.

| | | Equivalent |
|---|---|---|
| | | Glucose |
| | | Concentration |
| Board | Interference Layer Composition and Deposition | at 2 mg/dL |
| ID | Temperature | acetaminophen |
| V0051 | 1.5% EVA 12% VA, in xylene heated at 60° C. | 12.9 |
| UNF | 1% EVA 25% VA, in xylene at 23° C. | 16.6 |
| UNF | 1% EVA 40% VA, in xylene at 23° C. | 147 |

The data in the TABLE clearly indicates that the layers comprising reduced levels of wt. % vinyl acetate monomeric units effectively blocked/attenuated the known interferent acetaminophen as indicated by the calculated equivalent glucose concentration. Thus, based on the data of the TABLE and extrapolation, a layer of about 33 wt. % vinyl acetate monomeric units or less is considered effective as an interferent-reducing layer, and a layer of about 25 wt. % vinyl acetate monomeric units or less is considered particularly effective as an interferent-reducing layer. Moreover, the combination of an interferent-reducing layer comprising vinyl acetate monomeric units of about 33 wt. % or less in combination with an outer coating of a flux limiting layer of about 40 wt. % vinyl acetate or more provides a functional biosensor suitable for in vivo or in vitro use. Specifically, a combination of an interferent-reducing layer comprising poly (ethylene-co-vinyl acetate) with 25 wt. % or less vinyl acetate content in combination with an outer coating of a flux limiting layer of poly (ethylene-co-vinyl acetate) with about 40 wt. % vinyl acetate content provides a functional biosensor particularly suitable for in vivo or in vitro use.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification may be to be understood as being modified in all instances by the term “about.” Accordingly, unless indicated to the contrary, the numerical parameters set forth herein may be approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials. These descriptions are susceptible to modifications in the methods and materials, as well as alterations in the fabrication methods and equipment. Such modifications will become apparent to those skilled in the art from a consideration of this disclosure or practice of the disclosure. Consequently, it is not intended that this disclosure be limited to the specific embodiments disclosed herein, but that it cover all modifications and alternatives coming within the true scope and spirit of the claims.

What is claimed is:

1. An electrochemical analyte sensor comprising:
at least one electrode having an electroactive surface;
at least one interferent-reducing layer comprising vinyl ester monomeric units disposed distally from the electroactive surface;
an enzyme layer disposed distally from the electroactive surface; and.
a flux limiting membrane disposed over the at least one interferent-reducing layer and the enzyme layer.

2. A sensor of claim 1, wherein the at least one interferent-reducing layer is disposed more distally from the electroactive surface than the enzyme layer.

3. A sensor of claim 1, wherein the enzyme layer is disposed more distally from the electroactive surface than the at least one interferent-reducing layer.

4. A sensor of claim 1, wherein the enzyme layer is positioned between at least two interferent-reducing layers, at least one interferent layer comprising vinyl ester monomeric units.

5. A sensor of any one of the claims 1-4, wherein the at least one interferent-reducing layer is a poly (ethylene-co-vinyl acetate).

6. A sensor of claim 5, wherein the at least one interferent-reducing layer comprises a wt. % vinyl acetate content of 33 or less.

7. A sensor of any one of the claims 1-4, wherein the at least one interferent-reducing layer comprises:
a mixture of poly (ethylene-co-vinyl acetate) polymers, independently having a wt. % vinyl acetate content of about 12 and about 18;
a mixture of poly (ethylene-co-vinyl acetate) polymers, independently having a wt. % vinyl acetate content of about 12 and about 25;
a mixture of poly (ethylene-co-vinyl acetate) polymers, independently having a wt. % vinyl acetate content of about 18 and about 25;
a mixture of poly (ethylene-co-vinyl acetate) polymers, independently having a wt. % vinyl acetate content of about 12 and 33;
a mixture of poly (ethylene-co-vinyl acetate) polymers, independently having a wt. % vinyl acetate content of about 18 and 33;
a mixture of poly (ethylene-co-vinyl acetate) polymers, independently having a wt. % vinyl acetate content of about 25 and 33; or
a mixture of poly (ethylene-co-vinyl acetate) polymers, independently having a wt. % vinyl acetate content of 33 or less.

8. A sensor of any one of the claims 1-4, wherein the at least one interferent-reducing layer comprises a poly (ethylene-co-vinyl acetate) having a wt. % vinyl acetate content of about 18 or less.

9. A sensor of any one of the claims 1-4, further comprising a reference electrode, a counter electrode, an auxiliary electrode, or combinations thereof, having disposed thereon at least one anti-fouling layer.

10. A sensor of any one of the claims 1-4, wherein the enzyme layer comprises a hydrophilic polymer-enzyme composition.

11. A sensor of any one of the claims 1-4, wherein the enzyme layer comprises a hydrophilic polymer-glucose oxidase composition, wherein the hydrophilic polymer-enzyme composition comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N-N-dimethylacrylamide, polyacrylamide, and copolymers thereof.

12. A sensor of claim 11, wherein the flux limiting membrane is selected from polyurethanes, vinyl polymers, polyethers, polyesters, polyamides, rack-etched polycarbonates, polysiloxanes, polycarbosiloxanes, celluloses, protein-based materials, and mixtures or combinations thereof.

13. A sensor of claim 11, wherein the flux limiting membrane is a poly (ethylene-co-vinyl acetate) comprising about 40 wt. % vinyl acetate.

14. An electrochemical analyte sensor comprising:
at least one electrode having an electroactive surface;
a poly (ethylene-co-vinyl acetate) layer disposed distally from the electroactive surface, the poly (ethylene-co-vinyl acetate) having a wt. % vinyl acetate content of 33 or less;
an enzyme layer disposed distally from the poly (ethylene-co-vinyl acetate) having a wt. % vinyl acetate content of 33 or less, wherein the enzyme layer comprises a mixture of glucose oxidase and poly-N-vinylpyrrolidone; and
a poly (ethylene-co-vinyl acetate) layer having a wt. % vinyl acetate content of more than 33 disposed over the enzyme layer.

15. A method of measuring an analyte in a subject, the method comprising:
providing a electrochemical analyte sensor comprising:
(i) at least one electrode having an electroactive surface;
(ii) at least one interferent-reducing layer comprising vinyl ester monomeric units disposed distally from the electroactive surface;
(iii) an enzyme layer disposed distally from the electroactive surface; and
(iv) a flux limiting layer disposed over the at least one interferent-reducing layer and the enzyme layer;
contacting the electrochemical analyte sensor with a sample from a subject;
wherein an amount of interferent present in the sample reaching the electroactive surface is reduced.

16. A method of claim 15, wherein the at least one interferent-reducing layer comprising vinyl ester monomeric units is disposed more distally from the electroactive surface than the enzyme layer.

17. A method of claim 15, wherein the enzyme layer is disposed more distally from the electroactive surface than the at least one interferent-reducing layer.

18. A method of claim 15, wherein the enzyme layer is positioned between at least two interferent-reducing layers each comprising vinyl ester monomeric units.

19. A method of any one of the claims 15-18, wherein the at least one interferent-reducing layer is a poly (ethylene-co-vinyl acetate) having a wt. % vinyl acetate content of 33 or less.

20. A method of any one of the claims 15-18, wherein the at least one interferent-reducing layer is a poly (ethylene-co-vinyl acetate) having a wt. % vinyl acetate content of about 18 or less.

21. A method of any one of the claims 15-18, wherein the flux limiting membrane is a poly (ethylene-co-vinyl acetate) comprising about 40 wt. % vinyl acetate.

22. A method of any one of the claims 15-18, wherein the sample is in vivo intravenous blood of a subject.

23. A method of any one of the claims 15-18, wherein the electrochemical analyte sensor is disposed in a catheter.

24. A method of any one of the claims 15-18, further comprising a reference electrode, a counter electrode, an auxiliary electrode, or combinations thereof, having disposed thereon at least one anti-fouling layer.
